(19) United States
(12) Patent Application Publication
(10) Pub. No.: US 2025/0295659 A1
(43) Pub. Date: Sep. 25, 2025

(54) METHODS FOR TREATING TRAUMATIC BRAIN INJURY

(71) Applicant: The Regents of the University of California, Oakland, CA (US)

(72) Inventors: DaZhi Liu, Davis, CA (US); Bruce Lyeth, Davis, CA (US); Ryan Martin, Elk Grove, CA (US); Kiarash Shahlaie, Sacramento, CA (US)

(73) Assignee: The Regents of the University of California, Oakland, CA (US)

(21) Appl. No.: 18/712,594

(22) PCT Filed: Nov. 28, 2022
(86) PCT No.: PCT/US2022/051052

§ 371 Date: May 22, 2024

Related U.S. Application Data
(60) Provisional application No. 63/283,940, filed on Nov. 29, 2021.

(51) Int. Cl. A61K 31/506; A61P 25/14; A61P 25/28

(52) U.S. Cl. CPC A61K 31/506; A61P 25/14; A61P 25/28

(57) ABSTRACT

The disclosure provides compositions and methods for treating traumatic brain injury (TBI). The compositions comprise inhibitors of multiple protein tyrosine kinase families, including Src, Abl, and/or c-Kit protein tyrosine kinase families. The methods comprise administering a therapeutically effective amount of a composition comprising an inhibitor of multiple protein tyrosine kinase families to a subject, wherein the inhibitor is administered at a dose of less than about 20 mg/day to an adult human.

### CROSS-REFERENCE TO RELATED PATENT APPLICATIONS

This application is a US National Phase Under 371 of International Application PCT/US2022/051052 filed Nov. 28, 2022, which claims the benefit of priority to U.S. Provisional Patent Application No. 63/283,940, filed Nov. 29, 2021, the disclosures of which are hereby incorporated by reference in their entireties for all purposes.

### STATEMENT AS TO RIGHTS TO INVENTIONS MADE UNDER FEDERALLY SPONSORED RESEARCH AND DEVELOPMENT

This invention was made with Government support under Grant Nos. R01NS089901 and R01NS114061, awarded by the National Institutes of Health (NIH). The Government has certain rights in the invention.

### BACKGROUND

Traumatic brain injury (TBI) is a leading cause of death and disability¹, with 69 million individuals estimated to suffer this injury from all causes worldwide each year². The global TBI treatment market is expected to grow steadily from $112.6 billion in 2017 to $156.8 billion in 2024, at a compound annual growth rate (CAGR) of 4.8% from 2018 to 20243. Geographically, North America holds the major share of global TBI treatment market owing to the growing number of TBI patients, and the increasing research and development activity on diagnosis management³. However, there is currently no U.S. Food and Drug Administration (FDA) approved drug for treatment of TBI.

### BRIEF SUMMARY

The present disclosure provides compositions and methods that are useful for treating TBI.

In one aspect, a method of treating TBI in a human subject in need thereof is provided, the method comprising administering a therapeutically effective amount of a composition comprising an inhibitor of multiple kinase families to the subject, wherein the inhibitor is administered at a dose of less than or equal to about 20 mg/day.

In some embodiments, the inhibitor has an in vitro IC₅₀ of less than about 4 nM in cell-free assays.

In some embodiments, the inhibitor is selected from an inhibitor of a Src family kinase, an Abl family kinase, a c-Kit family kinase, or a combination thereof. In some embodiments, the inhibitor inhibits a Src-family tyrosine kinase selected from Src, Frk, Lck, Lyn, Blk, Hck, Fyn, Yrk, Fgr, Yes, or a combination thereof. In some embodiments, the inhibitor is an inhibitor of Src but not other Src-family tyrosine kinases. In some embodiments, the inhibitor is an inhibitor of Src but not Lck or Fyn.

In some embodiments, the inhibitor is dasatinib or a salt thereof.

In some embodiments, wherein the inhibitor has the structure of Formula I:

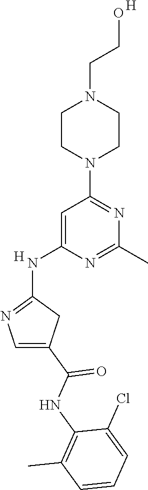

In some embodiments, the subject is an adolescent or adult human.

In some embodiments, the dose comprises about 10 mg/day to about 20 mg/day. In some embodiments, the subject is an adolescent or adult human, and the dose comprises about 10 mg/day to about 20 mg/day. In some embodiments, the inhibitor is administered in one or more doses. In some embodiments, the inhibitor is administered in a single dose. In some embodiments, the single dose comprises about 10 mg to about 20 mg. In some embodiments, the single dose comprises about 20 mg.

In some embodiments, the inhibitor is administered during the acute stage of TBI. In some embodiments, the inhibitor is administered between about 0.1 to about 9 hours following TBI. In some embodiments, the inhibitor is administered as single dose between about 0.1 to about 9 hours following TBI, and the dose comprises about 10 mg to about 20 mg. In some embodiments, the inhibitor is administered as single dose between about 0.1 to about 9 hours following TBI, and the dose comprises less than or equal to about 20 mg.

In some embodiments, the TBI is classified as mild, moderate or severe TBI. In some embodiments, the TBI is classified as mild TBI, or the subject is able to swallow, and the composition is administered to the subject orally. In some embodiments, the composition is administered to the subject as a tablet or pill. In some embodiments, the TBI is classified as moderate or severe TBI, or the subject is unconscious or otherwise unable to swallow, and the composition is administered to the subject intravenously. In some embodiments, the TBI is classified as moderate or severe TBI, or the subject is unconscious or otherwise unable to swallow, and the composition is administered to the subject intracranially.

In some embodiments, the composition comprises an inhibitor that can cross the blood-brain barrier (BBB). For example, the composition can comprise a Src/Abl/c-Kit inhibitor that crosses the BBB. In some embodiments, the Src/Abl/c-Kit inhibitor that crosses the BBB is dasatinib or ponatinib. In some embodiments, the composition comprising an inhibitor that can cross the BBB is administered to a subject classified with mild TBI who has an intact BBB. Thus, in situations where the subject is classified with mild TBI and/or there is no associated bleeding (indicating the BBB is intact), the subject can be administered an inhibitor that crosses the BBB.

In subjects having a TBI that presents with bleeding or other indication where the BBB is disrupted (no longer intact), the inhibitor does not need to cross the BBB and can reach the site of injury where the BBB is disrupted. Thus, in situations where the subject is classified with moderate or severe TBI associated with bleeding, the subject can be administered an inhibitor that is not capable of crossing the BBB. In some embodiments, the inhibitor that does not cross the BBB is imatinib, an Abl/c-KIt inhibitor.

In some embodiments, the composition comprises an inhibitor that can inhibit systemic inflammation (e.g., platelet aggregation, platelet-leukocyte complex adhesion, or leukocyte infiltration). By reducing systemic inflammation, the inhibitor can reduce BBB disruption after TBI. For example, the composition can comprise a Src/Abl/c-Kit inhibitor that can inhibit systemic inflammation and protect the brain after TBI, regardless if the inhibitor can cross the BBB.

In some embodiments, the composition has decreased side effects compared to a dose administered at greater than 100 mg/day. In some embodiments, the side effect is selected from Table 5. In some embodiments, the side effects are selected from the group consisting of muscle pain, weakness, joint pain, pain, burning or tingling in the hands or the feet, rash, skin redness, peeling skin, swelling, redness, pain inside the mouth, mouth sores, diarrhea, nausea, vomiting, constipation, stomach pain, loss of appetite, weight loss, and a combination thereof.

In some embodiments, the composition improves cognitive function in the subject as measured by the Montreal Cognitive Assessment, the Overall Test Battery Mean, a latent ability composite score, the Neuropsychological Deficit Score, or a combination thereof.

In some embodiments, administering the composition to the subject results in decreased symptoms of TBI as determined by the Glasgow outcome scale extended (GOSE). In some embodiments, administering the composition to the subject results in a 2 level improvement on the GOSE at 6 months after the TBI occurred.

In some embodiments, administering the composition to the subject results in decreased symptoms of TBI selected from the group consisting of headache, nausea or vomiting, fatigue or drowsiness, speech problems, dizziness or loss of balance, blurred vision, ringing in the ears, a bad taste in the mouth, changes in the ability to smell, sensitivity to light or sound, loss of consciousness, confusion, disorientation, memory or concentration problems, mood changes or mood swings, depression, anxiety, difficulty sleeping, sleeping more than usual, convulsions or seizures, dilation of one or both pupils of the eyes, clear fluids draining from the nose or ears, inability to awaken from sleep, weakness or numbness in fingers and toes, loss of coordination, agitation, combativeness, slurred speech, coma, and a combination thereof.

In some embodiments, administering the composition to the subject results in decreased symptoms of TBI-induced brain damage selected from the group consisting of intracranial hemorrhage, brain hematoma, motor deficits, disruption of the blood brain barrier, brain edema, and a combination thereof.

In some embodiments, administering the composition to the subject results in decreased symptoms of primary or secondary TBI selected from the group consisting of accumulation of intracellular calcium in neurons, cell depolarization, excitotoxic release of glutamate, disruption of ionic gradients, impaired mitochondrial function, elevated reactive oxygen species, neuroinflammation, and a combination thereof.

In some embodiments, the composition further comprises a pharmaceutically acceptable carrier or excipient.

In another aspect, use of a composition comprising an inhibitor of the disclosure for treating TBI is provided. In some embodiments, a composition comprising an inhibitor of a Src family kinase, an Abl family kinase, a c-Kit family kinase, or a combination thereof for use as a medicament is provided. In some embodiments, the medicament is for use in treating TBI. In some embodiments, a composition comprising an inhibitor of a Src family kinase, an Abl family kinase, a c-Kit family kinase, or a combination thereof for use in the treatment of TBI is provided.

### BRIEF DESCRIPTION OF THE DRAWINGS

FIG. 1 shows hemorrhage distributes in ipsilateral parenchyma and bilateral ventricles at 24 hrs post-TBI. Scale bars: 3.25 mm.

FIG. 2 shows dasatinib reduces spatial memory deficits 11 through 15 days after TBI in rats. The Y-axis shows latency to finding platform (sec). Each point represents the mean±standard error. Green line: vehicle treated TBI (TBI/vehicle); red line: dasatinib treated TBI (TBI/dasatinib); blue line: sham control (sham/Vehicle). *P<0.05 vs TBI rats treated with vehicle; **P<0.01 vs sham treated rats (Repeated measures ANOVA followed by Dunnett's post hoc test).

FIGS. 3A-3J show that dasatinib decreases CA2-3 neuronal loss at 24 hrs after CCI-TBI in rats. FIGS. 3A and 3B show the results of a control sham operation (not TBI). FIGS. 3C and 3D show the results of TBI in control rats treated with vehicle. FIGS. 3E and 3F show the results of rats treated with dasatinib at 0 hrs post TBI. FIGS. 3G and 3H show the results of rats treated with dasatinib at 3 hrs post TBI. FIGS. 3I and 3J show the results of rats treated with dasatinib at 6 hrs post TBI. 2 mg/kg of dasatinib was injected intraperitoneally (i.p.) at 0, 3, or 6 hrs post-TBI. Scale bars: 200 μm in A,C,E,G,I; 50 μm in B,D,F,H,J.

FIG. 4 shows that a single acute intraperitoneal administration of dasatinib (2 mg/kg, administered i.p. at 0, 3, or 6 hrs post-TBI) significantly promotes survival of CA2-3 neurons 24 hrs after CCI-TBI in rats. *P<0.05, **P<0.01 vs. CCI-TBI rats treated with vehicle.

FIG. 5 shows that dasatinib (2 mg/kg, administered i.p. at 0, 3, or 6 hrs post-TBI) mitigates motor imbalance at 22-24 hrs after CCI-TBI in rats. *P<0.05 vs. CCI-TBI rats treated with vehicle.

### DETAILED DESCRIPTION

### Definitions

As used herein, the term “brain injury” refers to a direct or indirect damage to the brain or head. A brain injury may be caused by a direct or indirect physical damage to the brain or head (i.e., a fall, an assault, or a motor vehicle accident). A brain injury may also be caused by a disease that is directly or indirectly related to the brain or head (i.e., meningitis). A brain injury may be confirmed to one area of the brain or head or involve more than one area of the brain or head. Some symptoms of brain injury include, but are not limited to, neuroinflammation, hypotension, hypoxia, edema, abnormalities in glucose utilization, cellular metabolism, membrane fluidity, synaptic function, and structural integrity of the brain. In some embodiments, a brain injury is an acute brain injury, e.g., traumatic brain injury (TBI), concussion, intracerebral hemorrhage (ICH), intraventricular hemorrhage (IVH), subarachnoid hemorrhage (SAH), seizure, and ischemic stroke.

As used herein, the terms “subject,” “individual,” and “patient” are used interchangeably herein to refer to a vertebrate, a mammal, or a human. Mammals include, but are not limited to, murines, rats, simians, humans, farm animals, sport animals, and pets. Tissues, cells and their progeny of a biological entity obtained in vivo or cultured in vitro are also encompassed.

As used herein, the term “administering” includes oral administration, topical contact, administration as a suppository, intravenous, intraperitoneal, intramuscular, intralesional, intrathecal, intranasal, intraosseous, or subcutaneous administration to a subject. Administration is by any route, including parenteral and transmucosal (e.g., buccal, sublingual, palatal, gingival, nasal, vaginal, rectal, or transdermal). Parenteral administration includes, e.g., intravenous, intramuscular, intra-arterial, intradermal, subcutaneous, intraperitoneal, intraventricular, intraosseous, and intracranial. Other modes of delivery include, but are not limited to, the use of liposomal formulations, intravenous infusion, transdermal patches, etc. In particular embodiments of the methods described herein, the inhibitor may be administered to the subject orally.

As used herein, the term “treating” refers to an approach for obtaining beneficial or desired results including, but not limited to, a therapeutic benefit and/or a prophylactic benefit. By therapeutic benefit is meant any therapeutically relevant improvement in or effect on one or more diseases, conditions, or symptoms under treatment. Therapeutic benefit can also mean to effect a cure of one or more diseases, conditions, or symptoms under treatment. For prophylactic benefit, the compositions may be administered to a subject at risk of developing a particular disease, condition, or symptom, or to a subject reporting one or more of the physiological symptoms of a disease, even though the disease, condition, or symptom may not have yet been manifested. In particular embodiments, beneficial results that may be obtained from the methods for treating a brain injury in a subject described herein include, e.g., prevention or reduction of lymphocyte infiltration (e.g., T cell infiltration), prevention or reduction of blood-brain barrier (BBB) disruption, and prevention or reduction of neuronal death.

As used herein, the term “therapeutically effective amount” refers to an amount, e.g., pharmaceutical dose, effective in inducing a desired biological effect in a subject or patient or in treating a patient having a condition or disorder described herein. It is also to be understood herein that a “therapeutically effective amount” may be interpreted as an amount giving a desired therapeutic effect, either taken in one dose or in any dosage or route, taken alone or in combination with other therapeutic agents. A therapeutically effective amount may be an amount that treats, prevents, alleviates, abates, or reduces the severity of symptoms of diseases and disorders (e.g., a brain injury).

As used herein, the term “pharmaceutical composition” refers to a medicinal or pharmaceutical formulation that contains an active ingredient as well as one or more excipients and diluents to enable the active ingredient suitable for the method of administration. The pharmaceutical composition of the present disclosure includes pharmaceutically acceptable components that are compatible with an inhibitor of the disclosure. The pharmaceutical composition may be in aqueous form for intravenous or subcutaneous administration or in tablet or capsule form for oral administration.

As used herein, the term “pharmaceutically acceptable carrier” refers to an excipient or diluent in a pharmaceutical composition. The pharmaceutically acceptable carrier must be compatible with the other ingredients of the formulation and not deleterious to the recipient. The nature of the carrier differs with the mode of administration. For example, for intravenous administration, an aqueous solution carrier is generally used; for oral administration, a solid carrier can be used.

The term “IC₅₀” refers to half-maximal inhibitory concentration, and is the most widely used and informative measure of a drug's efficacy. It indicates how much of a drug or compound is needed to inhibit a biological process by half, thus providing a measure of potency of an antagonist drug. The IC₅₀ of a pharmacological compound can be determined based on assays that utilize whole cell systems or by surface plasmon resonance to accurately determine IC₅₀ values of individual inhibitor-target pairs.

All numerical ranges disclosed herein include the endpoints at each end of the range, unless otherwise excluded, and all numerical values in between the endpoints, to the first significant digit. For example, a range of 1 to 10 can include the values 1.0, 1.1, 1.2, 1.3, . . . 9.7, 9.8, 9.9 and 10.0.

The term “about,” when modifying a numerical value of the disclosure, refers to values that include normal experimental variation in the art, and includes values that are plus or minus 1% to 10% of a recited value, e.g., +/−1%, 2%, 3%, 4%, 5%, 6%, 7%, 8% 9%, or 10%. All numerical values and ranges are understood to be modified by the term “about” regardless of whether or not the term “about” is expressly used to modify the value.

### INTRODUCTION

The present disclosure provides compositions and methods for treating traumatic brain injury (TBI). The compositions comprise inhibitors of one of more (or multiple) protein tyrosine kinase families. In some embodiments, the inhibitor is an inhibitor of a Src, Abl and/or c-Kit protein tyrosine kinase family. In some embodiments, the inhibitor is administered to a human subject suffering from TBI. While inhibitors of multiple protein tyrosine kinase families have been approved by the FDA to treat cancer and tumors, inhibitors of protein tyrosine kinase families s have not been approved by the FDA for treating TBI. While the FDA has approved the administration of Src kinase inhibitors to human cancer patients at relatively high doses, for example greater than 100 mg/day to an adult human, there are no reports of treating TBI with relatively low doses of kinase inhibitors, such as less than about 100 milligrams (mg)/day.

The compositions and methods of the disclosure provide advantages over existing treatments for TBI. For example, chronic administration of protein tyrosine kinase inhibitors at higher doses typically administered for treating cancer (e.g., equal to or greater than 100 mg/day) can result in undesirable side effects, including muscle pain, weakness, joint pain, pain, burning or tingling in the hands or the feet, rash, skin redness, peeling skin, swelling, redness, pain inside the mouth, mouth sores, diarrhea, nausea, vomiting, constipation, stomach pain, loss of appetite, weight loss, or combinations thereof. See the internet at medlineplus.gov/druginfo/meds/a607063.html; and lls.org/leukemia/chronic-myeloid-leukemia/treatment/side-effects. Thus, by administering the composition to human patients at relatively low doses (e.g., less than 100 mg/day), the undesirable side effects associated with higher doses can be reduced.

In addition, protein tyrosine kinase inhibitors are often administered to cancer patients chronically (over time such as days, weeks or months), which can cause long-term inhibition of kinases (e.g., Src, Abl). Long term inhibition of kinases can result in additional undesirable side effects, as these kinases play important roles in neurogenesis, blood-brain barrier self-repair, and other important cellular processes during the recovery phase after TBI. Therefore, the compositions described herein can be administered as a single dose during the acute phase of TBI, rather than chronically over time, which should further reduce the undesirable side effects associated with chronic administration of protein tyrosine kinase inhibitors.

### Methods

The methods of the disclosure are useful for treating TBI in a subject, such as a human. In some embodiments, the methods comprise administering a therapeutically effective amount of a composition comprising an inhibitor of a Src, Abl, or c-Kit protein tyrosine kinase to the subject. The inhibitor can be administered at a dose of less than about 100 mg/day, e.g. at a dose of less than 100 mg, less than 90 mg, less than 80 mg, less than 70 mg, less than 60 mg, less than 50 mg, less than 40 mg, less than 30 mg, less than 20 mg or less than 10 mg/day. In some embodiments, the inhibitor can be administered at a dose of about 10 to about 30 mg/day, or a single dose of about 10 to about 30 mg. The dose administered is typically determined based on the weight of the subject, and the values provided herein are based on a 60 kg human. One of ordinary skill in the art can adjust the dose based on the body surface area, weight or mass of an individual subject. See, e.g., Nair A B, Jacob S. A simple practice guide for dose conversion between animals and human. J Basic Clin Pharm. 2016; 7(2):27-31.

The Src family of kinases are membrane-associated, non-receptor tyrosine kinases that play a role in many signal transduction pathways in the cell, and regulate a variety of cellular processes, including cell proliferation, differentiation, apoptosis, migration, and metabolism. The Src kinase family includes 10 members: Src (Proto-oncogene tyrosine-protein kinase Src, also known as proto-oncogene c-Src, or c-Src (cellular Src)), Frk, Lck, Lyn, Blk, Hck, Fyn, Yrk, Fgr, and Yes. The SrcA subfamily includes Src, Yes, Fyn and Fgr. The SrcB subfamily includes Lck, Hck, Blk, and Lyn. FRK (Fyn-related kinase) is in a separate subfamily. Src family kinases contain six conserved domains: a N-terminal myristoylated segment, a SH2 domain, a SH3 domain, a linker region, a tyrosine kinase domain, and C-terminal tail (see Parsons S J, Parsons J T (October 2004). “Src family kinases, key regulators of signal transduction”. Oncogene. 23 (48): 7906-9.) Inhibitors of Src family kinases have been developed to treat cancer, but no Src kinase inhibitors have been approved by the FDA to treat TBI.

In some embodiments the Src kinase inhibitor is selected from PP121 (CAS No. 1092788-83-4), Ponatinib (AP24534; CAS No. 943319-70-8), Bosutinib (SKI-606; CAS No. 380843-75-4), KX2-391 (Tirbanibulin; CAS No. 897016-82-9), Saracatinib (AZD0530; CAS No. 379231-04-6), Dasatinib (BMS-354825; CAS No. 302962-49-8), Dasatinib hydrochloride (BMS-354825; CAS No. 854001-07-3), Dasatinib Monohydrate (BMS-354825; CAS No. 863127-77-9), ENMD-2076 (CAS No. 934353-76-1), XL228 (CAS No. 898280-07-4), DGY-06-116 (CAS No. unknown), TPX-0022 (CSF1R-IN-2; CAS No. 2271119-26-5), eCF506 (CAS No. 1914078-41-3), Src Inhibitor 1 (CAS No. 179248-59-0), UM-164 (CAS No. 903564-48-7), Repotrectinib (TPX-0005; CAS No. 1802220-02-5), CCT196969 (CAS No. 1163719-56-9), WH-4-023 (KIN001-112, KIN112, Dual LCK/SRC inhibitor; CAS No. 837422-57-8), Dehydroabietic acid (DAA, DHAA; CAS No. 1740-19-8), Ginkgolic acid C17:1 (GAC 17:1; CAS No. 111047-30-4), AD80 (CAS No. 1384071-99-1), and combinations thereof.

In some embodiments the Src kinase inhibitor is selected from dasatinib (BMS-354825; CAS No. 302962-49-8), dasatinib hydrochloride (BMS-354825; CAS No. 854001-07-3), dasatinib monohydrate (BMS-354825; CAS No. 863127-77-9), and combinations thereof. In some embodiments, the inhibitor has the structure of Formula I:

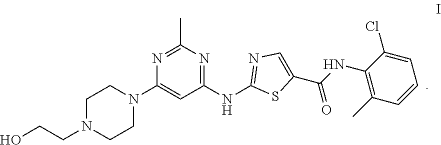 [SMILES: Cc1nc(Nc2ncc(C(=O)Nc3c(C)cccc3Cl)s2)cc(N2CCN(CCO)CC2)n1]

In some embodiments, the inhibitor inhibits a Src-family tyrosine kinase. In some embodiments, the inhibitor inhibits a Src-family tyrosine kinase selected from Src, Frk, Lck, Lyn, Blk, Hck, Fyn, Yrk, Fgr, Yes, or a combination thereof.

In some embodiments, the inhibitor is an inhibitor of Src but does not inhibit other Src-family tyrosine kinases. Thus, in some embodiments, the inhibitor inhibits Src, but does not inhibit Frk, Lck, Lyn, Blk, Hck, Fyn, Yrk, Fgr, Yes, or a combination thereof. In some embodiments, the inhibitor inhibits Src, but not Lck and/or Fyn. In some embodiments, the Src inhibitor also inhibits one or more additional Src family kinases. Thus, in some embodiments, the inhibitor inhibits Src and one or more of Frk, Lck, Lyn, Blk, Hck, Fyn, Yrk, Fgr, and Yes, or a combination thereof.

In some embodiments, the Src inhibitor has an IC50 of less than 100 nM (e.g., less than 100 nM, less than 90 nM, less than 80 nM, less than 70 nM, less than 60 nM, less than 50 nM, less than 40 nM, less than 30 nM, less than 20 nM, less than 10 nM, less than 5 nM, less than 4 nM, less than 3 nM, less than 2 nM, or less than 1 nM), in cell free assays.

Abl-family tyrosine kinases are conserved nonreceptor tyrosine kinases that contain SH3-SH2-TK (Src homology 3-Src homology 2-tyrosine kinase) domain cassette. The SH3-SH2-TK domain cassette is coupled to an actin-binding domain. The Abl tyrosine kinases are involved in a variety of cellular processes, including cell division, adhesion, differentiation, and response to stress. The Abl-family tyrosine kinases include proto-oncogene Abl1, ABL2 (also known as Abl-related gene or Arg), c-Abl (the endogenous mammalian gene), and v-Abl (viral Abl). In chronic myelogenous leukemia, a translocation results in a fusion between the BCR and ABL1 genes, resulting in BCR-ABL.

In some embodiments the Abl kinase inhibitor is selected from Table 1 below, including combinations thereof:

TABLE 1
Abl Inhibitors.

Dasatinib (BMS-354825)
Imatinib (STI571) Mesylate
Tozasertib (VX-680, MK-0457)
Ponatinib (AP24534)
Imatinib (STI571)
Nilotinib (AMN-107)
Bosutinib (SKI-606)
Danusertib (PHA-739358)
AT9283
Degrasyn (WP1130)
Bafetinib (INNO-406)
KW-2449
NVP-BHG712
PP121
Nocodazole (R17934)
Rebastinib (DCC-2036)
Olverembatinib dimesylate (HQP1351)
GNF-2
1-NM-PP1
Nilotinib hydrochloride monohydrate
XL228
1-Naphthyl PP1(1-NA-PP1)
Nilotinib hydrochloride
Dasatinib hydrochloride
Asciminib (ABL001)
GNF-7
Radotinib
Dasatinib Monohydrate
GNF-5
URMC-099
PD173955
GMB-475
AST-487 (NVP-AST487)
Berbamine
Berbamine dihydrochloride

In some embodiments, the Abl kinase inhibitor inhibits one or more of Abl, Bcr-Abl, c-Abl, or v-Abl, or combinations thereof.

In some embodiments the Abl kinase inhibitor is selected from dasatinib (BMS-354825; CAS No. 302962-49-8), dasatinib hydrochloride (BMS-354825; CAS No. 854001-07-3), dasatinib monohydrate (BMS-354825; CAS No. 863127-77-9), and combinations thereof.

In some embodiments, the Abl kinase inhibitor has an IC50 of less than 100 nM (e.g., less than 100 nM, less than 90 nM, less than 80 nM, less than 70 nM, less than 60 nM, less than 50 nM, less than 40 nM, less than 30 nM, less than 20 nM, less than 10 nM, or less than 1 nM), in cell free assays.

c-Kit is a type III receptor tyrosine kinase that is encoded by the proto-oncogene c-Kit. The ligand for c-Kit is stem cell factor (SCF). c-Kit is the cellular homolog of the v-kit oncogene.

In some embodiments the c-Kit kinase inhibitor is selected from Table 2 below, including combinations thereof.

TABLE 2
c-Kit Inhibitors.

Sorafenib (BAY 43-9006) tosylate
Dasatinib (BMS-354825)
Imatinib (STI571) Mesylate
Axitinib (AG 013736)
Imatinib (STI571)
Foretinib (GSK1363089)
Regorafenib (BAY 73-4506)
Pazopanib Hcl (GW786034 HCl)
Cediranib (AZD2171)
Dovitinib (TKI-258)
Masitinib (AB1010)
Tivozanib (AV-951)
Amuvatinib (MP-470)
Motesanib Diphosphate (AMG-706)
Lenvatinib (E7080)
OSI-930
Ki8751
Telatinib
Pazopanib
Cabozantinib malate (XL184)
Tyrphostin AG 1296
Agerafenib (RXDX-105)
Regorafenib Hydrochloride
UNC2025
Ki20227
SU14813
CS-2660 (JNJ-38158471)
Ripretinib (DCC-2618)
Lenvatinib (E7080) Mesylate
Regorafenib (BAY-734506) Monohydrate
Avapritinib (BLU-285)
Sitravatinib (MGCD516)
Pexidartinib (PLX3397)
Dasatinib Monohydrate
Dovitinib (TKI258) Lactate
AZD2932
Sunitinib (SU11248) malate
SU5614
ISCK03
AST-487 (NVP-AST487)
AZD3229
PDGFR inhibitor 1
Erdafitinib (JNJ-42756493)
Sunitinib (SU11248)

In some embodiments the c-Kit kinase inhibitor is selected from dasatinib (BMS-354825; CAS No. 302962-49-8), dasatinib hydrochloride (BMS-354825; CAS No. 854001-07-3), dasatinib monohydrate (BMS-354825; CAS No. 863127-77-9), and combinations thereof.

In some embodiments, the c-Kit kinase inhibitor has an IC50 of less than 100 nM (e.g., less than 100 nM, less than 90 nM, less than 80 nM, less than 70 nM, less than 60 nM, less than 50 nM, less than 40 nM, less than 30 nM, less than 20 nM, less than 10 nM, or less than 1 nM), in cell free assays.

In some embodiments, the subject is a human. In some embodiments, the subject is an adult human. In some embodiments, the subject is an adolescent human.

In some embodiments, the subject is an adolescent or adult human, and the dose of the inhibitor comprises less than 100 mg/day, e.g. at a dose of less than 100 mg, less than 90 mg, less than 80 mg, less than 70 mg, less than 60 mg, less than 50 mg, less than 40 mg, less than 30 mg, less than 20 mg or less than 10 mg/day. In some embodiments, the subject is an adolescent or adult human, and the inhibitor can be administered at a dose of about 10 mg/day to about 100 mg/day, about 10 mg/day to about 90 mg/day, about 10 mg/day to about 80 mg/day, about 10 mg/day to about 70 mg/day, about 10 mg/day to about 60 mg/day, about 10 mg/day to about 50 mg/day, about 10 mg/day to about 40 mg/day, about 10 mg/day to about 30 mg/day, about 10 mg/day to about 20 mg/day, about 20 mg/day to about 100 mg/day, about 20 mg/day to about 90 mg/day, about 20 mg/day to about 80 mg/day, about 20 mg/day to about 70 mg/day, about 20 mg/day to about 60 mg/day, about 20 mg/day to about 50 mg/day, about 20 mg/day to about 40 mg/day, and about 20 mg/day to about 30 mg/day.

In some embodiments, the subject is an adolescent or adult human, and the dose of the inhibitor comprises about 10 mg/day to about 30 mg/day (e.g., about 10, 11, 12, 13, 14, 15, 16, 17, 18, 19, 20, 21, 22, 23, 24, 25, 26, 27, 28, 29 or 30 mg/day). In some embodiments, the subject is an adolescent or adult human, and the dose of the inhibitor comprises about 20 mg/day.

In some embodiments, the composition comprising the inhibitor is administered in one or more doses. Thus, in embodiments where the subject is an adolescent or adult human, the composition is administered in one or more doses comprising a dose of inhibitor of about 10 mg/day to about 30 mg/day (e.g., about 10, 11, 12, 13, 14, 15, 16, 17, 18, 19, 20, 21, 22, 23, 24, 25, 26, 27, 28, 29 or 30 mg/day). In some embodiments, the subject is an adolescent or adult human, and composition is administered in one or more doses comprising a dose of inhibitor of about 20 mg/day. The composition can be administered one or more times per day, daily, weekly, or monthly. In some embodiments, the composition is administered during the acute phase of TBI. In some embodiments, the composition is administered during the chronic phase of TBI.

In some embodiments, the composition is administered in a single dose. In some embodiments, the subject is an adolescent or adult human, and the composition is administered in a single dose of about 10 mg to about 100 mg, about 10 mg to about 90 mg, about 10 mg to about 80 mg, about 10 mg to about 70 mg, about 10 mg to about 60 mg, about 10 mg to about 50 mg, about 10 mg to about 40 mg, about 10 mg to about 30 mg, about 10 mg to about 20 mg, about 20 mg to about 100 mg, about 20 mg to about 90 mg, about 20 mg to about 80 mg, about 20 mg to about 70 mg, about 20 mg to about 60 mg, about 20 mg to about 50 mg, about 20 mg to about 40 mg, and about 20 mg to about 30 mg.

In some embodiments, the composition is administered in a single dose during the acute phase of TBI. In some embodiments, the composition is administered in a single dose between about 0.1 to about 9 hours following TBI (e.g., about 0.1, 0.2, 0.3, 0.4, 0.5, 1, 2, 3, 4, 5, 6, 7, 8, or 9 hours following TBI). In some embodiments, the subject is an adolescent or adult human, and the composition is administered in a single dose of about 10 mg to about 100 mg, about 10 mg to about 90 mg, about 10 mg to about 80 mg, about 10 mg to about 70 mg, about 10 mg to about 60 mg, about 10 mg to about 50 mg, about 10 mg to about 40 mg, about 10 mg to about 30 mg, about 10 mg to about 20 mg, about 20 mg to about 100 mg, about 20 mg to about 90 mg, about 20 mg to about 80 mg, about 20 mg to about 70 mg, about 20 mg to about 60 mg, about 20 mg to about 50 mg, about 20 mg to about 40 mg, and about 20 mg to about 30 mg during the acute phase of TBI. In some embodiments, the subject is an adolescent or adult human, and the composition is administered in a single dose of about 10 mg to about 100 mg, about 10 mg to about 90 mg, about 10 mg to about 80 mg, about 10 mg to about 70 mg, about 10 mg to about 60 mg, about 10 mg to about 50 mg, about 10 mg to about 40 mg, about 10 mg to about 30 mg, about 10 mg to about 20 mg, about 20 mg to about 100 mg, about 20 mg to about 90 mg, about 20 mg to about 80 mg, about 20 mg to about 70 mg, about 20 mg to about 60 mg, about 20 mg to about 50 mg, about 20 mg to about 40 mg, and about 20 mg to about 30 mg between about 0.1 to about 9 hours following TBI (e.g., about 0.1, 0.2, 0.3, 0.4, 0.5, 1, 2, 3, 4, 5, 6, 7, 8, or 9 hours following TBI).

In some embodiments, the composition is administered in a single dose of about 10 to about 30 mg ((e.g., about 10, 11, 12, 13, 14, 15, 16, 17, 18, 19, 20, 21, 22, 23, 24, 25, 26, 27, 28, 29 or 30 mg) between about 0.1 hour to about 9 hours following TBI. In some embodiments, the composition is administered in a single dose of about 20 mg between about 0.1 hour to about 9 hours following TBI.

As understood by a person of skill in the art, the dose of an inhibitor of the disclosure administered to a subject to treat TBI can be adjusted based on the compounds efficacy an potency. For example, inhibitor compounds having an in vitro IC₅₀ of less than 10 nM in cell-free assays can be administered at lower doses than compounds having an in vitro IC₅₀ of greater than 10 nM in cell-free assays. Thus, in some embodiments, inhibitor compounds having an in vitro IC₅₀ of less than 4 nM in cell-free assays can be administered at doses of 20 mg/day, or a single dose of 20 mg.

Representative IC₅₀ and doses for inhibitors of c-Src, Abl and c-Kit kinases are shown in Table 3 below:

| | | | Estimated | |
|---|---|---|---|---|
| Target | Drug/kinase | IC₅₀ (nm) | dose in | |
| kinase | inhibitor | in vitro | vivo | |
| c-Src | Dasatinib | 0.8 nm | 0.4~4 | mg/kg |
| | Bosutinib | 1.2 | 0.6-6 | mg/kg |
| | Ponatinib | 5.4 | 2.7-27 | mg/kg |
| Abl | Dasatinib | 0.6 | 0.3~3 | mg/kg |
| | Bosutinib | 1 | 0.5-5 | mg/kg |
| | Imatinib | 600 | 300-3000 | mg/kg |
| | Nilotinib | 30 | 15-150 | mg/kg |
| | Ponatinib | 0.37 | 0.2-4 | mg/kg |
| c-Kit | Dasatinib | 79 | 40-400 | mg/kg |
| | Avapritinib | 0.5 | 0.25-2.5 | mg/kg |
| | Cabozantinib | 4.6 | 2.3-23 | mg/kg |
| | Imatinib | 100 | 50-500 | mg/kg |
| | Lenvatinib | 100 | 50-500 | mg/kg |
| | Pazopanib | 74 | 37-370 | mg/kg |
| | Pexidartinib | 10 | 5-50 | mg/kg |
| | Ponatinib | 12.5 | 6-60 | mg/kg |
| | Regorafenib | 7 | 3.5-35 | mg/kg |
| | Ripretinib | 4 | 2-20 | mg/kg |
| | Sorafenib | 68 | 38 | mg/kg |
| | Sunitinib | 22 | 11-110 | mg/kg |

The TBI amendable to treatment by the methods of the disclosure can be classified as mild, moderate or severe TBI. In some embodiments, the TBI is classified as mild TBI (e.g., the subject has not lost consciousness or has regained consciousness), and the composition is administered to the subject orally, for example as a pill or tablet. Table 4 below shows a representative classification of TBI severity.

TABLE 4
Classification of TBI Severity.

| Criteria | Mild | | Moderate | Severe | |
|---|---|---|---|---|---|
| Structural imaging | Normal | | Normal or abnormal | Abnormal | |
| Loss of Consciousness (LOC) | 0-30 | min | >30 min and <24 hrs | >24 | hrs |
| Alteration of consciousness/mental state | less than or equal to | | >24 hours. Severity based on other | | |
| (AOC) | 24 hrs | | criteria | | |
| Post-traumatic amnesia (PTA) | 0-1 | day | >1 and <7 days | >7 | days |
| Glasgow Coma Scale (best available score in | 13-15 | | 9-12 | <9 | |
| first 24 hours) | | | | | |

### From: APPENDIX C, DEFINITION OF MTBI FROM THE VA/DOD CLINICAL PRACTICE GUIDELINE FOR MANAGEMENT OF CONCUSSION/MILD TRAUMATIC BRAIN INJURY (2009)

As noted above, the compositions of the disclosure can be administered at inhibitor doses that reduce the side effects associated with doses that are approved for treatment of cancer patients. Thus, in some embodiments, the composition has decreased side effects compared to a dose of inhibitor administered to a subject at greater than 100 mg/day. In some embodiments, the inhibitor is a Src kinase inhibitor, and the composition has decreased side effects compared to a dose of inhibitor administered to a subject at greater than 100 mg/day.

In some embodiments, the side effects are selected from the group consisting of muscle pain, weakness, joint pain, pain, burning or tingling in the hands or the feet, rash, skin redness, peeling skin, swelling, redness, pain inside the mouth, mouth sores, diarrhea, nausea, vomiting, constipation, stomach pain, loss of appetite, weight loss, and a combination thereof.

In some embodiments, the compositions of the disclosure are administered at inhibitor doses that reduce a side effect associated with a c-Src, Abl or c-Kit inhibitor shown in Table 5 below compared to a dose of inhibitor approved for treatment of cancer patients. In some embodiments, the compositions of the disclosure are administered at inhibitor doses that reduce a side effect associated with a c-Src, Abl or c-Kit inhibitor shown in Table 5 below compared to a dose of inhibitor administered to a subject at greater than 100 mg/day.

TABLE 5
Common side effects of tyrosine kinase inhibitors approved by the FDA.

| Target | Drug/kinase | Common Side effects when used at the doses for FDA-approved |
|---|---|---|
| kinase | inhibitor | treatments |
| c-Src | Dasatinib | Infection, Hypophosphatemia, Hypocalcemia, Body fluid retention, Anemia, |
| | | Thrombocytopenia disorder, Neutropenic disorder, Peripheral neuropathy, |
| | | Hemorrhage, Upper respiratory infection, Pleural effusions, Stomatitis, |
| | | Constipation, Gastrointestinal hemorrhage, Pruritus of skin, Arthralgia, Dizziness, |
| | | Fever, Fatigue, Malaise |
| | Bosutinib | Diarrhea, Nausea, Thrombocytopeniasorder, Vomiting, Kidney disease with |
| | | reduction in GFR, Skin rash, Anemia, Fever, Fatigue, Neutropenic disorder, |
| | | Abnormal hepatic function tests, Upper respiratory infection, Lower respiratory |
| | | infection, General weakness, Anorexia, Pharyngitis, Back pain, Headache disorder, |
| | | Dizziness, Dyspnea |
| | Ponatinib | Thromboembolic disorder, Arterial thrombosis, Hypertension, Thrombocytopenic |
| | | disorder, Neutropenic disorder, Anemia, Skin rash, Leukopenia, Acute abdominal |
| | | pain, Fatigue, Headache disorder, Dry skin, Constipation, Arthralgia, Nausea, |
| | | Fever, Lymphopenia, Bone marrow depression, Diarrhea, Vomiting |
| Abl | Dasatinib | Same as above |
| | Bosutinib | Same as above |
| | Imatinib | Body fluid retention, Anemia, Thrombocytopenia disorder, Neutropenic disorder, |
| | | Pericardial effusion, Hemorrhage, Upper respiratory infection, Gastrointestinal |
| | | hemorrhage, Arthralgia, Back pain, Myalgia, Cramps, Dizziness, Fever, Fatigue, |
| | | Skin rash, Edema, Nausea, Vomiting, Diarrhea |
| | Nilotinib | Hypocalcemia, Hyponatremia, Back pain, Hyperkalemia, Hypokalemia, |
| | | Pancytopenia, Anemia, Thrombocytopenia disorder, Leukopenia, Neutropenic |
| | | disorder, Elevated serum lipase, Hypertension, Prolonged QT interval, Pharyngitis, |
| | | Constipation, Muscle spasm, Pruritus of skin, Arthralgia, Myalgia, Vertigo |
| | Ponatinib | Same as above |
| c-Kit | Dasatinib | Same as above |
| | Avapritinib | Edema, Nausea, Fatigue, General weakness, Acute confusion, Vomiting, Anorexia, |
| | | Diarrhea, Hair discoloration, Eye tearing, Acute abdominal pain, Constipation, Skin |
| | | rash, Dizziness, Anemia, Leukopenia |
| | Cabozantinib | Blurred vision, burning, numbness, tingling, or painful sensations, confusion, dark |
| | | urine, darkening of the skin, decreased urination, diarrhea, difficulty with breathing |
| | | or swallowing, dizziness, dry mouth, fainting, headache, increase in heart rate, |
| | | lightheadedness, loss of appetite, mental depression, nausea or vomiting, |
| | | nervousness, pain in the chest, groin, or legs, especially the calves, pounding in the |
| | | ears, rapid breathing, redness, swelling, or pain of the skin, scaling of the skin on |
| | | the hands and feet, severe, sudden headache, skin rash or ulcers, slow or fast |
| | | heartbeat, slurred speech, stomach pain, sudden loss of coordination, sudden, |
| | | severe weakness or numbness in the arm or leg, sunken eyes, thirst, unsteadiness or |
| | | awkwardness, unusual tiredness or weakness, vision changes, weakness in the |
| | | arms, hands, legs, or feet, wrinkled skin, yellow eyes or skin |
| | Imatinib | Same as above |
| | Lenvatinib | Hypertension, Fatigue, Diarrhea, Arthralgia, Myalgia, Anorexia, Weight loss, |
| | | Nausea, Stomatitis, Headache disorder, Vomiting, Proteinuria, Palmar-plantar |
| | | erythrodysesthesia, Acute abdominal pain, Voice change, Epistaxis, Hemorrhage, |
| | | Hypothyroidism, Dyspnea, Increased alanine transaminase |
| | Pazopanib | Nausea, Vomiting, Diarrhea, Thrombocytopenia disorder, Leukopenia, Neutropenic |
| | | disorder, Alopecia, Hypertension, Fatigue, Hemorrhage, General weakness, Skin |
| | | rash, Proteinuria, Anorexia, Weight loss, Headache disorder, Hair bleaching, Pain, |
| | | Myalgia, Thrombotic disorder |
| | Pexidartinib | Blurred vision, decreased appetite, dizziness, fever, headache, itching or skin rash, |
| | | loss of appetite, nausea and vomiting, nervousness, pounding in the ears, slow or |
| | | fast heartbeat, swelling of the feet or lower legs |
| | Ponatinib | Same as above |
| | Regorafenib | Hypertension, Impaired wound healing, General weakness, Fatigue, Anorexia, |
| | | Palmar-plantar erythrodysesthesia, Diarrhea, Stomatitis, Weight loss, Infection, |
| | | Voice change, Hemorrhage, Fever, Skin rash, Headache disorder, |
| | | Hypophosphatemia, Pain, Nausea, Acute abdominal pain, Anemia |
| | Ripretinib | Alopecia, Fatigue, Nausea, Acute abdominal pain, Constipation, Myalgia, Diarrhea, |
| | | Anorexia, Palmar-plantar erythrodysesthesia, Vomiting, Elevated serum lipase, |
| | | Hypophosphatemia |
| | Sorafenib | Hypophosphatemia, Anemia, Thrombocytopenia disorder, Leukopenia, |
| | | Neutropenic disorder, Lymphopenia, Depression, Peripheral neuropathy, |
| | | Hypertension, Hemorrhage, Stomatitis, Dyspepsia, Constipation, Gastrointestinal |
| | | hemorrhage, Erectile dysfunction, Eczema, Exfoliative dermatitis, Erythema, |
| | | Pruritus of skin, Alopecia |
| | Sunitinib | Increased risk of bleeding, Pain in extremities, Peripheral edema, Back pain, |
| | | Hypertension, Chronic heart failure |
| | | Stomatitis, Dyspepsia, Constipation, Dry skin, Dyschromia, Arthralgia, Headache |
| | | disorder, Dizziness, Fever, Fatigue, General weakness, Dysgeusia, Skin rash, |
| | | Anorexia |

In some embodiments, the composition improves cognitive function in the subject as measured by the Montreal Cognitive Assessment, the Overall Test Battery Mean, a latent ability composite score, the Neuropsychological Deficit Score, or a combination thereof. See Silverberg N D, et al., Developing a Cognition Endpoint for Traumatic Brain Injury Clinical Trials. J Neurotrauma. 2017 Jan. 15; 34(2):363-371; Mishra K., et al. Montreal cognitive Assessment Score: A Screening Tool for Cognitive Function in Traumatic Brain Injury (TBI) Population. Journal of Neurology & Neuromedicine, 2020.

In some embodiments, administering the composition to the subject results in decreased symptoms of TBI as determined by the Glasgow outcome scale extended (GOSE). In some embodiments, administering the composition to the subject results in at least a 2 level improvement on the GOSE at 6 months after the TBI occurred. In some embodiments, administering the composition to the subject results in a 2 level, 3 level or 4 level improvement on the GOSE at 6 months after the TBI occurred.

The Glasgow Outcome Scale (GOS) is a global scale for functional outcome that rates patient status into one of five categories: Dead, Vegetative State, Severe Disability, Moderate Disability or Good Recovery. The Extended GOS (GOSE) provides more detailed categorization into eight categories by subdividing the categories of severe disability, moderate disability and good recovery into a lower and upper category, as shown in Table 6 below (see Jennett B, Bond M: Assessment of outcome after severe brain damage. Lancet 1:480-484, 1975; Teasdale G M, Pettigrew L E, Wilson J T, Murray G, Jennett B. Analyzing outcome of treatment of severe head injury: A review and update on advancing the use of the Glasgow Outcome Scale. Journal of Neurotrauma 1998; 15:587-597; Wilson J T L, Pettigrew L E L, Teasdale G M. Structured interviews for the Glasgow Outcome Scale and the Extended Glasgow Outcome Scale: Guidelines for Their Use. J Neurotrauma 15(8): 573-85. 1997; Wilson J T, Slieker F J, Legrand V, Murray G, Stocchetti N, Maas AI. Observer variation in the assessment of outcome in traumatic brain injury: experience from a multicenter, international randomized clinical trial. Neurosurgery. July; 61(1):123-8; discussion 128-9. 2007; Sander, A. (2002). The Extended Glasgow Outcome Scale. The Center for Outcome Measurement in Brain Injury. See the internet at tbims.org/combi/gose)

TABLE 6
Extended Glasgow Outcome Scale (GOSE)

| | | |
|---|---|---|
| 1. | Death (D) | |
| 2. | Vegetative State (VS) | (VS) Condition of unawareness with only |
| | | reflex responses but with periods of |
| | | spontaneous eye opening. |
| 3. | Low Severe Disability (SD−) | Patient who is dependent for daily support for |
| | | mental or physical disability, usually a |
| | | combination of both. If the patient can be left |
| | | alone for more than 8 h at home it is upper |
| | | level of SD, if not then it is low level of SD. |
| 4. | Upper Severe Disability | |
| | (SD+) | |
| 5. | Low Moderate Disability | Patients have some disability such as aphasia, |
| | (MD−) | hemiparesis or epilepsy and/or deficits of |
| | | memory or personality but are able to look |
| | | after themselves. They are independent at |
| | | home but dependent outside. If they are able |
| | | to return to work even with special |
| | | arrangement it is upper level of MD, if not |
| | | then it is low level of MD |
| 6. | Upper Moderate Disability | |
| | (MD+) | |
| 7. | Low Good Recovery (GR−) | Resumption of normal life with the capacity to |
| | | work even if pre-injury status has not been |
| | | achieved. Some patients have minor |
| | | neurological or psychological deficits. If these |
| | | deficits are not disabling then it is upper level |
| | | of GR, if disabling then it is lower level of |
| | | GR. |
| 8. | Upper Good Recovery (GR+) | |

* Use of the structured interview is recommended to facilitate consistency in ratings.

In some embodiments, the method results in a decrease in one or more symptoms of TBI. In some embodiments, administering a composition comprising an inhibitor of a Src, Abl, or c-Kit protein tyrosine kinase to the subject results in a decrease in one or more symptoms of TBI. In some embodiments, administering a composition comprising an inhibitor of a Src, Abl, or c-Kit protein tyrosine kinase to the subject at a dose of inhibitor less than about 100 mg/day (e.g., at a dose of less than 100 mg, less than 90 mg, less than 80 mg, less than 70 mg, less than 60 mg, less than 50 mg, less than 40 mg, less than 30 mg, less than 20 mg or less than 10 mg/day) results in a decrease in one or more symptoms of TBI. In some embodiments, the subject is an adolescent or adult human, and administering a composition comprising an inhibitor of a Src, Abl, or c-Kit protein tyrosine kinase to the subject in one or more doses comprising a dose of inhibitor of about 10 mg/day to about 30 mg/day (e.g., about 10, 11, 12, 13, 14, 15, 16, 17, 18, 19, 20, 21, 22, 23, 24, 25, 26, 27, 28, 29 or 30 mg/day) results in a decrease in one or more symptoms of TBI. In some embodiments, the subject is an adolescent or adult human, and administering a composition comprising an inhibitor of a Src, Abl, or c-Kit protein tyrosine kinase to the subject in one or more doses comprising a dose of inhibitor of about 20 mg/day results in a decrease in one or more symptoms of TBI. In some embodiments, the subject is an adolescent or adult human, and administering a composition comprising an inhibitor of a Src, Abl, or c-Kit protein tyrosine kinase to the subject in a single dose comprising a dose of inhibitor of about 10 mg to about 30 mg (e.g., about 10, 11, 12, 13, 14, 15, 16, 17, 18, 19, 20, 21, 22, 23, 24, 25, 26, 27, 28, 29 or 30 mg) results in a decrease in one or more symptoms of TBI. In some embodiments, the subject is an adolescent or adult human, and administering a composition comprising an inhibitor of a Src, Abl, or c-Kit protein tyrosine kinase to the subject in a single dose comprising a dose of inhibitor of about 20 mg results in a decrease in one or more symptoms of TBI.

In some embodiments, administering a composition of the disclosure to a subject results in a decrease in one or more symptoms of TBI selected from the group consisting of headache, nausea or vomiting, fatigue or drowsiness, speech problems, dizziness or loss of balance, blurred vision, ringing in the ears, a bad taste in the mouth, changes in the ability to smell, sensitivity to light or sound, loss of consciousness, confusion, disorientation, memory or concentration problems, mood changes or mood swings, depression, anxiety, difficulty sleeping, sleeping more than usual, convulsions or seizures, dilation of one or both pupils of the eyes, clear fluids draining from the nose or ears, inability to awaken from sleep, weakness or numbness in fingers and toes, loss of coordination, agitation, combativeness, slurred speech, coma, and a combination thereof. In some embodiments, administering a composition of the disclosure to a subject results in a decrease in one or more symptoms of TBI selected from the group consisting of intracranial hemorrhage, brain hematoma, motor deficits, disruption of the blood brain barrier, brain edema, and a combination thereof.

In some embodiments, administering a composition of the disclosure to a subject results in decreased symptoms of primary or secondary TBI selected from the group consisting of accumulation of intracellular calcium in neurons, cell depolarization, excitotoxic release of glutamate, disruption of ionic gradients, impaired mitochondrial function, elevated reactive oxygen species, neuroinflammation, and a combination thereof. Primary TBI refers to injury that occurs at the moment of initial trauma, and includes: skull fracture (breaking of the bony skull), contusions (bruise/bleed on the brain) that can lead to hematomas (blood clots in the meningeal layers or in the cortical/subcortical structures as a result of the trauma), concussions (low velocity injury resulting in functional deficits without pathological injury), lacerations (tears in brain tissue or blood vessels of the brain), diffuse axonal injury (traumatic shearing forces leading to tearing of nerve fibers in the white matter tracts).

Primary injuries can be caused by either a penetrating (open-head) injury or a nonpenetrating (closed-head) injury. A penetrating (open-head) injury involves an open wound to the head from a foreign object (e.g., bullet). It is typically marked by focal damage that occurs along the route the object has traveled in the brain that includes fractured/perforated skull, torn meninges, and damage to the brain tissue. A nonpenetrating (closed-head) injury is marked by brain damage due to indirect impact without the entry of any foreign object into the brain. The skull may or may not be damaged, but there is no penetration of the meninges. Nonpenetrating injuries can be of two types: acceleration and non-acceleration injuries. Acceleration injuries are caused by movement of the brain within the unrestrained head (e.g. whiplash injury). If the force impacting the head is strong enough, it can cause a contusion at the site of impact and the opposite side of the skull, causing an additional contusion (coup-contrecoup injury). Non-acceleration injuries are caused by injury to a restrained head and, therefore, no acceleration or deceleration of the brain occurs within the skull (e.g., blow to the head). These usually result in deformation (fracture) of the skull, causing focal localized damage to the meninges and brain.

Secondary injury occurs as an indirect result of the insult. It results from processes initiated by the initial trauma and typically evolves over time. Secondary injury includes ischemia (insufficient blood flow); hypoxia (insufficient oxygen in the brain); hypo/hypertension (low/high blood pressure); cerebral edema (swelling of the brain); raised intracranial pressure (increased pressure within the skull), which can lead to herniation (parts of the brain are displaced); hypercapnia (excessive carbon dioxide levels in the blood); meningitis (infection of the meningeal layers) and brain abscess; biochemical changes (changes in levels of neurotransmitters, sodium, potassium, etc.); epilepsy. See Hegde, M. N. (2006). A coursebook on aphasia and other neurogenic language disorders (3rd ed.). Clifton Park, NY: Delmar Cengage Learning.

### Pharmaceutical Compositions

The compositions of the disclosure include pharmaceutical compositions. The pharmaceutical compositions can include a compound or drug that is an inhibitor of a protein tyrosine kinase of the disclosure. In some embodiments, the compound or drug is an inhibitor of a protein tyrosine kinase selected from Src, Abl and/or c-Kit. In some embodiments, the compound or drug is an inhibitor of a protein tyrosine kinase selected from Src, Abl and c-Kit. In some embodiments, the inhibitor inhibits one or more of Src, Abl and c-Kit. In some embodiments, the compound or drug inhibits a Src-family tyrosine kinase. In some embodiments, the compound or drug inhibits a Src-family tyrosine kinase selected from Src, Frk, Lck, Lyn, Blk, Hck, Fyn, Yrk, Fgr, Yes, or a combination thereof. In some embodiments, the Src inhibitor also inhibits one or more additional Src family kinases. Thus, in some embodiments, the compound or drug inhibits Src and one or more of Frk, Lck, Lyn, Blk, Hck, Fyn, Yrk, Fgr, and Yes, or a combination thereof.

In some embodiments, pharmaceutical composition comprises a compound or drug that inhibits Src but does not inhibit other Src family members. Thus, in some embodiments, the compound or drug inhibits Src, but does not inhibit Frk, Lck, Lyn, Blk, Hck, Fyn, Yrk, Fgr, Yes, or a combination thereof. In some embodiments, the compound or drug does not inhibit Src family members Lck and Fyn. In some embodiments, the compound or drug inhibits Src, but not Lck and/or Fyn.

In some embodiments, the pharmaceutical composition comprises a compound or drug that inhibits a protein tyrosine kinase of the disclosure and has an IC50 of less than 100 nM (e.g., less than 100 nM, less than 90 nM, less than 80 nM, less than 70 nM, less than 60 nM, less than 50 nM, less than 40 nM, less than 30 nM, less than 20 nM, less than 10 nM, less than 5 nM, less than 4 nM, less than 3 nM, less than 2 nM, or less than 1 nM), in cell free assays.

In some embodiments, the pharmaceutical composition further comprises an excipient or pharmaceutically acceptable carrier. Pharmaceutically acceptable carriers and formulations are described in REMINGTON'S PHARMACEUTICAL SCIENCES, 23RD ED., Academic Press (2020). The pharmaceutical compositions may be administered as a formulation prepared in pharmaceutically acceptable diluents; for example, saline, phosphate buffer saline (PBS), aqueous ethanol, or solutions of glucose, mannitol, dextran, propylene glycol, oils (e.g., vegetable oils, animal oils, synthetic oils, etc.), microcrystalline cellulose, carboxymethyl cellulose, hydroxylpropyl methyl cellulose, magnesium stearate, calcium phosphate, gelatin, or polysorbate 80, or as solid formulations in appropriate excipients.

In some embodiments, a therapeutically effective amount of the pharmaceutical composition is administered to the subject in an amount sufficient to treat the brain injury (e.g., TBI), prevent or reduce neuronal death, prevent or reduce lymphocyte infiltration (e.g., T cell infiltration), and/or prevent or reduce BBB disruption after the brain injury (e.g., TBI).

In some embodiments, a therapeutically effective amount of the pharmaceutical composition comprises a dose of protein tyrosine kinase inhibitor of the disclosure less than about 100 mg/day, e.g., a dose of protein tyrosine kinase inhibitor less than 100 mg, less than 90 mg, less than 80 mg, less than 70 mg, less than 60 mg, less than 50 mg, less than 40 mg, less than 30 mg, less than 20 mg or less than 10 mg/day. In some embodiments, a therapeutically effective amount of the pharmaceutical composition comprises a dose of protein tyrosine kinase of the disclosure of about 10 mg to about 100 mg, about 10 mg to about 90 mg, about 10 mg to about 80 mg, about 10 mg to about 70 mg, about 10 mg to about 60 mg, about 10 mg to about 50 mg, about 10 mg to about 40 mg, about 10 mg to about 30 mg, about 10 mg to about 20 mg, about 20 mg to about 100 mg, about 20 mg to about 90 mg, about 20 mg to about 80 mg, about 20 mg to about 70 mg, about 20 mg to about 60 mg, about 20 mg to about 50 mg, about 20 mg to about 40 mg, and about 20 mg to about 30 mg. In some embodiments, a therapeutically effective amount of the pharmaceutical composition comprises a dose of protein tyrosine kinase inhibitor of about 10 mg/day to about 30 mg/day (e.g., about 10, 11, 12, 13, 14, 15, 16, 17, 18, 19, 20, 21, 22, 23, 24, 25, 26, 27, 28, 29 or 30 mg/day). In some embodiments, a therapeutically effective amount of the pharmaceutical composition comprises a dose of protein tyrosine kinase inhibitor of about 20 mg/day.

In some embodiments, a therapeutically effective amount of the pharmaceutical composition comprises a single dose of protein tyrosine kinase inhibitor less than about 100 mg, e.g., a dose of protein tyrosine kinase inhibitor less than 100 mg, less than 90 mg, less than 80 mg, less than 70 mg, less than 60 mg, less than 50 mg, less than 40 mg, less than 30 mg, less than 20 mg or less than 10 mg. In some embodiments, a therapeutically effective amount of the pharmaceutical composition comprises a single dose of protein tyrosine kinase inhibitor of about 10 mg to about 30 mg (e.g., about 10, 11, 12, 13, 14, 15, 16, 17, 18, 19, 20, 21, 22, 23, 24, 25, 26, 27, 28, 29 or 30 mg). In some embodiments, a therapeutically effective amount of the pharmaceutical composition comprises a single dose of protein tyrosine kinase inhibitor of about 20 mg. Determination of a therapeutically effective amount is within the capability of those skilled in the art.

In any of the above embodiments, the pharmaceutical composition can comprise a therapeutically effective amount of an inhibitor of a protein tyrosine kinase selected from Src, Abl and c-Kit.

### Routes and Administration

Pharmaceutical compositions of the disclosure comprising inhibitors of Src, Abl and/or c-Kit protein tyrosine kinases can be formulated for parenteral administration, e.g., intravenous administration, subcutaneous administration, intramuscular administration, intraarterial administration, intrathecal administration, intraperitoneal administration, or intracranial administration. In particular embodiments, the pharmaceutical composition may be formulated for oral administration, for example, as a tablet or pill. For injectable formulations, various effective pharmaceutical carriers are known in the art, see, e.g., ASHP Handbook on Injectable Drugs, Trissel, 18th ed. (2014). Other administration routes include, but are not limited to intravenous, rectal, transmucosal, intestinal, enteral, topical, suppository, through inhalation, intranasal, intraocular and intracranial administration.

In cases where the subject has mild TBI, or the subject is able to swallow, the pharmaceutical compositions comprising inhibitors of Src, Abl and/or c-Kit protein tyrosine kinases can be administered orally. In cases where the TBI does not disrupt the BBB, a pharmaceutical composition comprising inhibitors of Src, Abl and/or c-Kit protein tyrosine kinases that are capable of crossing the BBB can be administered to the subject.

Moderate and severe TBI is often accompanied by disruptions in the blood-brain barrier (BBB). In these cases, the pharmaceutical compositions comprising inhibitors of Src, Abl and/or c-Kit protein tyrosine kinases can be administered orally, if the subject is able to swallow, or by injection. In some embodiments, pharmaceutical compositions comprising inhibitors of Src, Abl and/or c-Kit protein tyrosine kinases can be administered intracranially at the site where the BBB is disrupted.

In some embodiments, administration may include a single dose or multiple doses. In some embodiments, pharmaceutical compositions for injection are presented in unit dosage form, e.g., in ampoules or in multi-dose containers. In some embodiments, a pharmaceutical composition described herein is administered in the form of a dosage unit (e.g., bolus).

The pharmaceutical compositions can be administered in a manner compatible with the dosage formulation and in such amount as is therapeutically effective to result in an improvement or remediation of the symptoms of TBI. In some embodiments, the dose is administered at intervals ranging from more than once per day, once per day, once per week, twice per week, three times per week, four times per week, five times per week, six times per week, once per month to once per three months, for as long as needed to sustain the desired effect. The timing between administrations may decrease as the medical condition improves or increase as the health of the patient declines. The dosage may be adapted by the physician in accordance with conventional factors such as the extent of the disease and different parameters of the subject.

### Kits

In another aspect, the disclosure provides a kit comprising an inhibitor of a protein tyrosine kinase of the disclosure. In some embodiments, the kit comprises an inhibitor of a Src, Abl, and/or c-Kit protein tyrosine kinase. The kit can also include one or more separate containers comprising an inhibitor of a Src, Abl, and/or c-Kit protein tyrosine kinase, along with written or computer readable instructions for administering the inhibitor to a subject having TBI.

### EXAMPLES

### Example 1

This example describes that treatment with a Src kinase inhibitor improves cognitive function in a representative experimental model of TBI.

### Methods

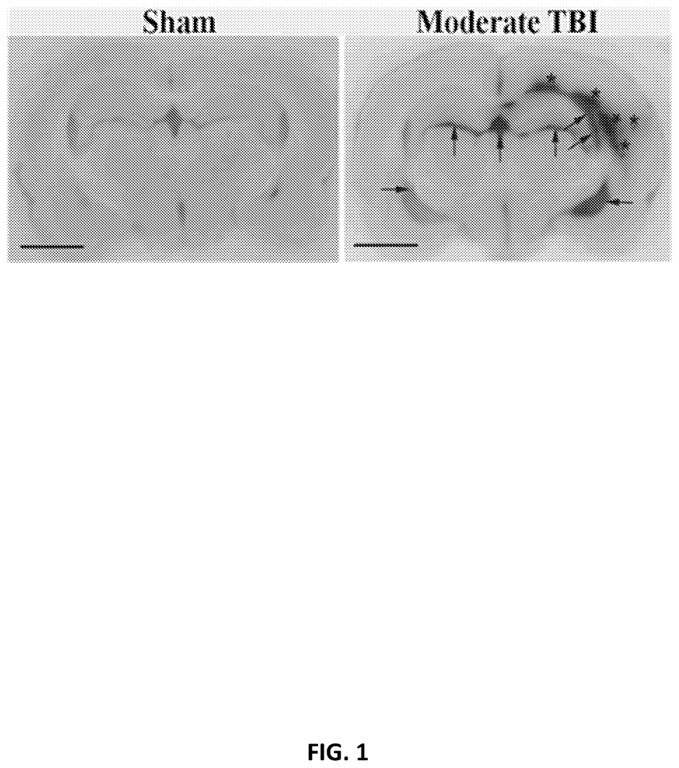

Moderate Lateral fluid percussion (LFP) was used to produce TBI in adult male Sprague-Dawley rats (weighing 300-350 grams). Brain contusion and hemorrhage were observed in ipsilateral parenchyma at 24 hr after TBI (See FIG. 1, marked with stars). Ventricular hemorrhage was also noted (marked with arrows in FIG. 1) and the amount of hemorrhage was greater in ipsilateral than contralateral ventricles (FIG. 1). Moreover, this model induced hippocampal neuron loss and memory deficits 16 days after TBI in rats. All of the following data were obtained using moderate LFP in adult rats.

A single acute administration of dasatinib after TBI. Dasatinib was dissolved in DMSO, diluted in 0.9% saline, and then administered intraperitoneally (2 mg/kg, i.p.) immediately after TBI in rats. Sham and vehicle control animals received 5% DMSO in 0.9% saline.

Morris Water Maze (MWM). Spatial memory was assessed using the MWM on days 12 through 16 after TBI. The apparatus and methods were previously described (14,23). In brief rats were released from one of four starting points and allowed 120 s to find the escape platform. Rats received a total of 4 trials per day, one from each starting point, over 5 consecutive days. Behavioral assessments were performed by an individual blinded to the treatment groups. Mean latency to find the platform was calculated for each day to assess learning as well as the mean swimming speed to assess motor function. Statistical differences were determined using repeated measures ANOVA followed by Dunnett's post hoc test.

Brain sample preparation and immunohistochemistry. Anesthetized rats were perfused with saline followed by 4% PFA at day 1 or day 16. Brain sections (50 μm) are cut at −20° C. and incubated in 0.3% H₂O₂ for 10 minutes to quench endogenous peroxidase, incubated with 3% horse blocking serum for 20 minutes, and then incubated overnight in primary antibody (mouse anti-NeuN, mature neuron marker, 1:150, Millipore), then in biotinylated secondary antibody (Goat anti-mouse 1:1,000, Vector) for 0.5 h, then placed in Vectastain ABC reagent for 0.5 hr and incubated in peroxidase substrate 3, 3′-diaminobenzidine (DAB) solution for 10 min.

Stereological cell counts. The total number of NeuN-positive neurons in CA2-3 of hippocampus were quantified bilaterally using stereological methods. The NeuN-positive cell counts were performed on a microscope (Nikon E600, Nikon) using commercial software (Stereo Investigator™8.0, Microbrightfield, Inc.). Serial sections cut at 50 μm thick were collected at Bregma −2.80 mm to Bregma −4.16 mm for a total of 10 sections per brain. Estimated numbers of the NeuN-positive neurons in the target brain region were generated by the Stereologer software using the following equation: ΣNobj=(ΣN)(1/SSF)(1/ASF)(1/TSF) (ref. 24). In this equation ΣNobj indicates the sum of objects sampled in the sections, SSF indicates the section sampling fraction, ASF indicates the area sampling fraction, and TSF indicates the thickness sampling fraction. Counting is performed by a blinded observer. Statistical differences are determined using ANOVA followed by Dunnett's post hoc test.

### Results

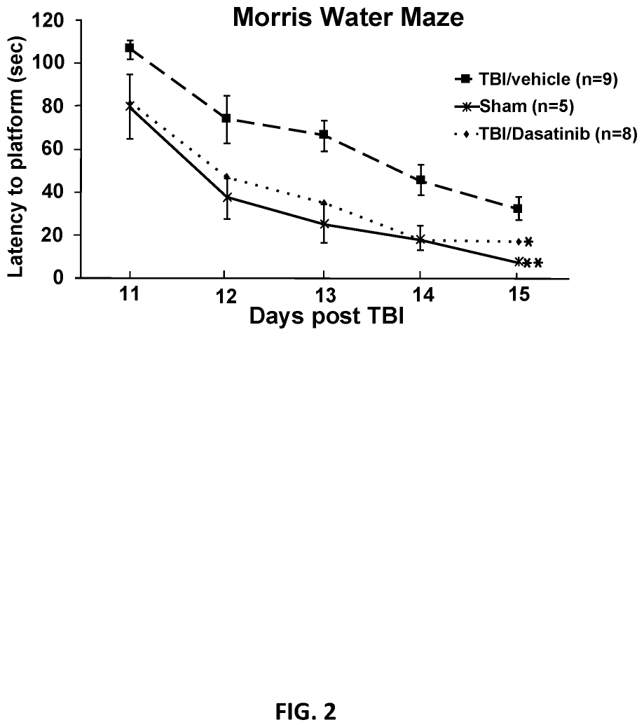

Dasatinib improves cognitive function after TBI in rats. The data shows that there was a significant effect of group on latency to find the platform in the Morris Water Maze (MWM) 12 through 16 days after TBI (F(2,19)=6.6, P<0.01; FIG. 2). The TBI/vehicle group had significantly longer latencies to find the hidden platform compared to the sham group (P<0.01) (FIG. 2). Treatment with dasatinib (2.0 mg/kg, i.p.) significantly improved performance compared to the vehicle treated TBI group (P<0.05) (FIG. 2). The MWM results suggested that dasatinib can improve cognitive function after TBI in rats.

### Example 2

This example describes a representative method for measuring IC₅₀ values of the inhibitors of the disclosure.

### Method for In Vitro Cell-Free Peptide Substrate Phosphorylation Assays.

The effects of the kinase inhibitors on the catalytic activity of unphosphorylated glutathione S-transferase (GST)-Abl kinase were assessed using a synthetic, NH2-terminal biotin-linked peptide substrate (biotin-EAIYAAPFAKKK-amide). Assays were carried out at 30° C. for 5 minutes in 25 μL of reaction mixture consisting of kinase buffer (25 mmol/L Tris-HCl (pH 7.5), 5 mmol/L β-glycerophosphate, 2 mmol/L DTT, 0.1 mmol/L Na3VO4, 10 mmol/L MgCl2; Cell Signaling Technology), 50 μmol/L peptide substrate, 10 nmol/L wild-type or mutant GST-Abl kinase, and 50 μmol/L ATP/[γ-32P]ATP (5,000 cpm/pmol). Reactions were terminated by addition of guanidine hydrochloride to a final concentration of 2.5 mol/L. A portion of each terminated reaction mixture was transferred to a streptavidin-coated membrane (SAM² biotin capture membrane; Promega, Madison, WI), washed, and dried according to the manufacturer's instructions; phosphate incorporation was determined by scintillation counting. Results were corrected for background binding to the membranes as determined by omitting peptide substrate from the kinase reaction. Time course experiments to establish the linear range of enzymatic activity preceded the kinase assays. To measure the IC₅₀ of Src family kinases, all conditions were as described above for GST-Abl kinase, except that a different biotinylated peptide substrate (SignaTECT PTK biotinylated peptide substrate 2 (Promega)) was used. See Cancer Res. 2005; 65(11):4500-4505. The IC₅₀ of Dasatinib was measured at a concentration of 0-32 nmol/L.

### Example 3

This example describes that treatment with a Src kinase inhibitor decreases neuronal loss in the hippocampus in a representative experimental model of TBI.

Methods: One day after controlled cortical impact (CCI)-TBI, rats were anesthetized and transcardially perfused with PBS followed by 4% PFA. Fixed brain coronal sections (50 μm) were cut and stained with NeuN (a mature neuron marker). The NeuN⁺ cells in ipsilateral hippocampal CA2-3 were quantified (n=4-8). Dasatinib (2 mg/kg) was administered as a single acute intravenous dose at 0, 3, or 6 hrs post-TBI.

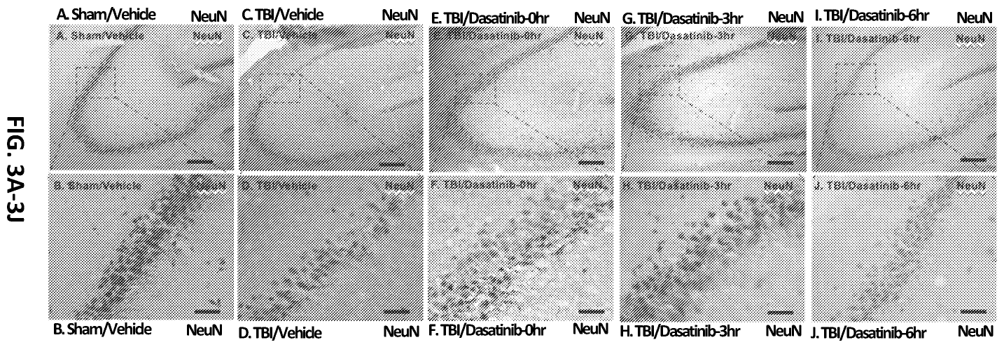

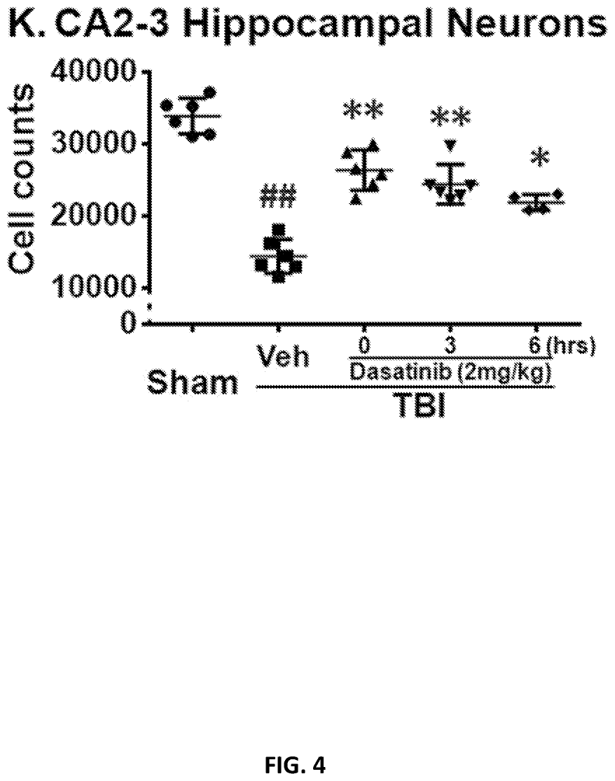

Results: As shown in FIG. 3 and FIG. 4, TBI causes loss of neurons in the ipsilateral side of TBI one day after TBI (^{##}P<0.01 vs. sham, FIGS. 3A-D, and FIG. 4), while a single acute intravenous administration of dasatinib significantly promotes survival of CA2-3 neurons 24 hrs after TBI in rats (*P<0.05, **P<0.01 vs TBI/Vehicle, FIGS. 3C-J, and FIG. 4).

### Example 4

This example describes that treatment with a Src kinase inhibitor mitigates motor imbalance after TBI.

Methods: Rats were subjected to elevated body swing test (BEST) at 24 hrs after TBI. Dasatinib (2 mg/kg) was administered as a single dose i.p. at 0 or 3 hrs post-TBI (n=4-8).

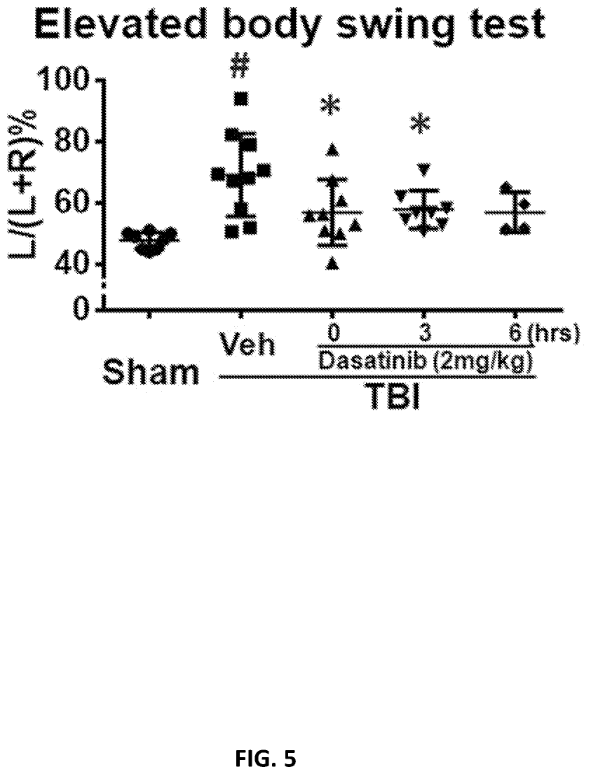

Results: As shown in FIG. 5, TBI causes biased swinging to the contralateral side of TBI 24 hrs after CCI-TBI (#P<0.05 vs. sham control). A single treatment of dasatinib (2.0 mg/kg, i.p.) administered at 0 or 3 hrs post-TBI significantly mitigated motor imbalance at 24 hrs after CCI-TBI in rats (*P<0.05 vs. TBI/vehicle, FIG. 5).

### REFERENCES

1 Faul, M., Xu, L., Wald, M. M. & Coronado, V. G. Traumatic brain injury in the United States: emergency department visits, hospitalizations, and deaths. Atlanta (GA): Centers for Disease Control and Prevention, National Center for Injury Prevention and Control. 7-23 (2010).
2 Dewan, M. C. et al. Estimating the global incidence of traumatic brain injury. Journal of neurosurgery, 1-18, doi:10.3171/2017.10.JNS17352 (2018).
3 EnergiasMarketResearch. Traumatic Brain Injuries Treatment Market. https://www.energiasmarketresearch.com/global-traumatic-brain-injuries-treatment-market-size/ (2018).
4 Liu, D. Z. & Ander, B. P. Cell cycle phase transitions: signposts for aberrant cell cycle reentry in dying mature neurons. Journal of Cytology & Histology 2, e101 (2011).
5 Liu, D. Z. & Ander, B. P. Cell cycle inhibition without disruption of neurogenesis is a strategy for treatment of aberrant cell cycle diseases: an update. ScientificWorldJoumal 2012, 491737 (2012).
6 Liu, D. Z., Ander, B. P. & Sharp, F. R. Cell cycle inhibition without disruption of neurogenesis is a strategy for treatment of central nervous system diseases. Neurobiology of disease 37, 549-557 (2010).
7 Summy, J. M. & Gallick, G. E. Src family kinases in tumor progression and metastasis. Cancer Metastasis Rev 22, 337-358 (2003).
8 Matsuoka, T. & Yashiro, M. Rho/ROCK signaling in motility and metastasis of gastric cancer. World J Gastroenterol 20, 13756-13766, doi:10.3748/wjg.v20.i38.13756 (2014).
9 Shi, Y. et al. Rapid endothelial cytoskeletal reorganization enables early blood-brain barrier disruption and long-term ischaemic reperfusion brain injury. Nat Commun 7, 10523, doi:10.1038/ncomms10523 (2016).
10 Liu, D. Z. et al. Blood-brain barrier breakdown and repair by Src after thrombin-induced injury. Annals of neurology 67, 526-533, doi:10.1002/ana.21924 (2010).
11 Putzke, A. P. et al. Metastatic progression of prostate cancer and e-cadherin regulation by zebl and SRC family kinases. Am J Pathol 179, 400-410, doi:10.1016/j.ajpath.2011.03.028 (2011).
12 Wu, Z. et al. Autophagy Blockade Sensitizes Prostate Cancer Cells towards Src Family Kinase Inhibitors. Genes Cancer 1, 40-49, doi:10.1177/1947601909358324 (2010).
13 Nam, J. S., Ino, Y., Sakamoto, M. & Hirohashi, S. Src family kinase inhibitor PP2 restores the E-cadherin/catenin cell adhesion system in human cancer cells and reduces cancer metastasis. Clin Cancer Res 8, 2430-2436 (2002).
14 Liu, D. et al. Inhibition of Src Family Kinases Protects Hippocampal Neurons and Improves Cognitive Function after Traumatic Brain Injury. Journal of neurotrauma 31, 1268-1276, doi:10.1089/neu.2013.3250 (2014).
15 Liu, D. Z. et al. Src kinase inhibition decreases thrombin-induced injury and cell cycle re-entry in striatal neurons. Neurobiology of disease 30, 201-211 (2008).
16 Roskoski, R., Jr. Src protein-tyrosine kinase structure, mechanism, and small molecule inhibitors. Pharmacol Res 94, 9-25, doi:10.1016/j.phrs.2015.01.003 (2015).
17 Yu, W. et al. Oxidation of KCNB1 Potassium Channels Causes Neurotoxicity and Cognitive Impairment in a Mouse Model of Traumatic Brain Injury. J Neurosci 36, 11084-11096, doi:10.1523/JNEUROSCI.2273-16.2016 (2016).
18 Nair, A. B. & Jacob, S. A simple practice guide for dose conversion between animals and human. J Basic Clin Pharm 7, 27-31, doi:10.4103/0976-0105.177703 (2016).
19 Medicine, U. S. N. L. o. MedlinePlus Drug Information: Dasatinib (2018).
20 Nosengo, N. Can you teach old drugs new tricks?Nature 534, 314-316, doi:10.1038/534314a (2016).
21 McIntosh, T. K. et al. Traumatic brain injury in the rat: characterization of a lateral fluid-percussion model. Neuroscience 28, 233-244 (1989).
22 Wang, T. et al. Effect of fish oil supplementation in a rat model of multiple mild traumatic brain injuries. Restorative neurology and neuroscience 31, 647-659, doi:10.3233/RNN-130316 (2013).
23 Lee, L. L., Galo, E., Lyeth, B. G., Muizelaar, J. P. & Berman, R. F. Neuroprotection in the rat lateral fluid percussion model of traumatic brain injury by SNX-185, an N-type voltage-gated calcium channel blocker. Experimental neurology 190, 70-78, doi:S0014-4886(04)00275-4 [pii]10.1016/j.expneurol.2004.07.003 (2004).
24 O'Hare, T., Walters, D. K., Stoffregen, E. P., Jia, T., Manley, P. W., Mestan, J., Cowan-Jacob, S. W., Lee, F. Y., Heinrich, M. C., Deininger, M. W. N., Druker, B. J. In vitro activity of Bcr-Abl inhibitors AMN107 and BMS-354825 against clinically relevant imatinib-resistant Abl kinase domain mutants. Cancer Res. 65(11), 4500-4505. doi: 10.1158/0008-5472.CAN-05-0259 (2005).

The embodiments illustrated and discussed in this specification are intended only to teach those skilled in the art the best way known to the inventors to make and use the invention. Nothing in this specification should be considered as limiting the scope of the present invention. All examples presented are representative and non-limiting. The above-described embodiments of the invention may be modified or varied, without departing from the invention, as appreciated by those skilled in the art in light of the above teachings. It is therefore to be understood that, within the scope of the claims and their equivalents, the invention may be practiced otherwise than as specifically described. All publications, patents, and patent applications cited in this specification are herein incorporated by reference as if each individual publication, patent, or patent application were specifically and individually indicated to be incorporated by reference.

### Exemplary Embodiments

Exemplary embodiments provided in accordance with the presently disclosed subject matter include, but are not limited to, the claims and the following embodiments:

1. A method of treating traumatic brain injury (TBI) in a human subject in need thereof, comprising administering a therapeutically effective amount of a composition comprising an inhibitor of multiple kinase families to the subject, wherein the inhibitor is administered at a dose of less than or equal to about 20 mg/day.
2. The method of embodiment 1, wherein the inhibitor has an in vitro IC50 of less than about 4 nM in cell-free assays.
3. The method of embodiment 1 or 2, wherein the inhibitor is selected from an inhibitor of a Src family kinase, an Abl family kinase, a c-Kit family kinase, or a combination thereof.
4. The method of embodiment 3, wherein the inhibitor inhibits a Src-family tyrosine kinase selected from Src, Frk, Lck, Lyn, Blk, Hck, Fyn, Yrk, Fgr, Yes, or a combination thereof.
5. The method of embodiment 4, wherein the inhibitor is an inhibitor of Src but not other Src-family tyrosine kinases.
6. The method of embodiment 5, wherein the inhibitor is an inhibitor of Src but not Lck or Fyn.
7. The method of any one of embodiments 1 to 6, wherein the inhibitor is dasatinib or a salt thereof.
8. The method of any one of embodiments 1 to 7, wherein the inhibitor has the structure of Formula I:

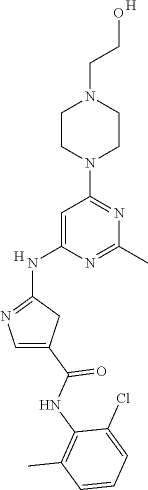

9. The method of any one of embodiments 1 to 8, wherein the subject is an adolescent or adult human.
10. The method of any one of embodiments 1 to 9, wherein the dose comprises about 10 mg/day to about 20 mg/day.
11. The method of any one of embodiments 1 to 10, wherein the inhibitor is administered in one or more doses.
12. The method of any one of embodiments 1 to 10, wherein the inhibitor is administered in a single dose.
13. The method of embodiment 12, wherein the inhibitor is administered during the acute stage of TBI.
14. The method of embodiment 12, wherein the inhibitor is administered between about 0.1 to about 9 hours following TBI.
15. The method of embodiment 12 or 13, wherein the single dose comprises about 10 mg to about 20 mg.
16. The method of any one of embodiments 12 to 15, wherein the single dose comprises about 20 mg.
17. The method of any one of embodiments 1 to 16, wherein the TBI is classified as mild, moderate or severe TBI.
18. The method of embodiment 17, wherein the TBI is classified as mild TBI, and the composition is administered to the subject orally.
19. The method of any one of embodiments 1 to 18, wherein the composition has decreased side effects compared to a dose administered at greater than 100 mg/day.
20. The method of embodiment 18, wherein the side effect is selected from Table 5.
21. The method of embodiment 19 or 20, wherein the side effects are selected from the group consisting of muscle pain, weakness, joint pain, pain, burning or tingling in the hands or the feet, rash, skin redness, peeling skin, swelling, redness, pain inside the mouth, mouth sores, diarrhea, nausea, vomiting, constipation, stomach pain, loss of appetite, weight loss, and a combination thereof.
22. The method of any one of embodiments 1 to 21, wherein the composition improves cognitive function in the subject as measured by the Montreal Cognitive Assessment, the Overall Test Battery Mean, a latent ability composite score, the Neuropsychological Deficit Score, or a combination thereof.
23. The method of any one of embodiments 1 to 22, wherein administering the composition to the subject results in decreased symptoms of TBI as determined by the Glasgow outcome scale extended (GOSE).
24. The method of embodiment 23, wherein administering the composition to the subject results in a 2 level improvement on the GOSE at 6 months after the TBI occurred.
25. The method of any one of embodiments 1 to 24, wherein administering the composition to the subject results in decreased symptoms of TBI selected from the group consisting of headache, nausea or vomiting, fatigue or drowsiness, speech problems, dizziness or loss of balance, blurred vision, ringing in the ears, a bad taste in the mouth, changes in the ability to smell, sensitivity to light or sound, loss of consciousness, confusion, disorientation, memory or concentration problems, mood changes or mood swings, depression, anxiety, difficulty sleeping, sleeping more than usual, convulsions or seizures, dilation of one or both pupils of the eyes, clear fluids draining from the nose or ears, inability to awaken from sleep, weakness or numbness in fingers and toes, loss of coordination, agitation, combativeness, slurred speech, coma, and a combination thereof.
26. The method of any one of embodiments 1 to 25, wherein the composition results in decreased symptoms of TBI-induced brain damage selected from the group consisting of intracranial hemorrhage, brain hematoma, motor deficits, disruption of the blood brain barrier, brain edema, and a combination thereof.
27. The method of any one of embodiments 1 to 26, wherein the composition results in decreased symptoms of primary or secondary TBI selected from the group consisting of accumulation of intracellular calcium in neurons, cell depolarization, excitotoxic release of glutamate, disruption of ionic gradients, impaired mitochondrial function, elevated reactive oxygen species, neuroinflammation, and a combination thereof.
28. The method of any one of embodiments 1 to 27, wherein the composition further comprises a pharmaceutically acceptable carrier or excipient.
29. The method of any one of embodiments 1 to 28, wherein the composition is administered to the subject as a tablet or pill.
30. The method of embodiment 17, wherein the TBI is classified as moderate or severe TBI, or the subject cannot swallow, and the composition is administered to the subject intravenously.
31. The method of embodiment 17, wherein the TBI is classified as moderate or severe TBI, or the subject cannot swallow, and the composition is administered to the subject intracranially.

1. A method of treating traumatic brain injury (TBI) in a human subject in need thereof, comprising administering a therapeutically effective amount of a composition comprising an inhibitor of multiple kinase families to the subject, wherein the inhibitor is administered at a dose of less than or equal to about 20 mg/day.

2. The method of claim 1, wherein the inhibitor has an in vitro IC₅₀ of less than about 4 nM in cell-free assays.

3. The method of claim 1, wherein the inhibitor is selected from an inhibitor of a Src family kinase, an Abl family kinase, a c-Kit family kinase, or a combination thereof.

4. The method of claim 3, wherein the inhibitor inhibits a Src-family tyrosine kinase selected from Src, Frk, Lck, Lyn, Blk, Hck, Fyn, Yrk, Fgr, Yes, or a combination thereof.

5. The method of claim 4, wherein the inhibitor is an inhibitor of Src but not other Src-family tyrosine kinases.

6. The method of claim 5, wherein the inhibitor is an inhibitor of Src but not Lck or Fyn.

7. The method of claim 1, wherein the inhibitor is dasatinib or a salt thereof.

8. (canceled)

9. (canceled)

10. The method of claim 1, wherein the dose comprises about 10 mg/day to about 20 mg/day.

11. The method of claim 1, wherein the inhibitor is administered in one or more doses.

12. The method of claim 1, wherein the inhibitor is administered in a single dose.

13. The method of claim 12, wherein the inhibitor is administered during the acute stage of TBI.

14. The method of claim 12, wherein the inhibitor is administered between about 0.1 to about 9 hours following TBI.

15. The method of claim 12, wherein the single dose comprises about 10 mg to about 20 mg.

16. The method of claim 12, wherein the single dose comprises about 20 mg.

17. The method of claim 1, wherein the TBI is classified as mild, moderate or severe TBI.

18. The method of claim 17, wherein the TBI is classified as mild TBI, and the composition is administered to the subject orally.

19. The method of claim 1, wherein the composition has decreased side effects compared to a dose administered at greater than 100 mg/day.

20-27. (canceled)

28. The method of claim 1, wherein the composition further comprises a pharmaceutically acceptable carrier or excipient.

29. The method of claim 1, wherein the composition is administered to the subject as a tablet or pill.

30. The method of claim 17, wherein the TBI is classified as moderate or severe TBI, or the subject cannot swallow, and the composition is administered to the subject intravenously or intracranially.

31. (canceled)
